(19) United States
(12) Patent Application Publication
(10) Pub. No.: US 2025/0079016 A1
(43) Pub. Date: Mar. 6, 2025

(54) ENHANCED VISION-BASED VITALS MONITORING USING MULTI-MODAL SOFT LABELS

(71) Applicant: SAMSUNG ELECTRONICS CO., LTD., Suwon-si (KR)

(72) Inventors: Korosh VATANPARVAR, San Jose, CA (US); Jeremy Speth, South Bend, IN (US); Nafiul Rashid, San Jose, CA (US); Li Zhu, Saratoga, CA (US); Migyeong Gwak, Santa Clara, CA (US); Jilong Kuang, San Jose, CA (US); Jun Gao, Menlo Park, CA (US)

(73) Assignee: SAMSUNG ELECTRONICS CO., LTD., Suwon-si (KR)

(21) Appl. No.: 18/814,096

(22) Filed: Aug. 23, 2024

Related U.S. Application Data
(60) Provisional application No. 63/536,675, filed on Sep. 5, 2023.

(51) Int. Cl. G16H 50/30; G06V 10/25; G06V 10/32; G06V 10/82; G06V 40/16; G16H 50/20

(52) U.S. Cl. CPC G16H 50/30; G06V 10/25; G06V 10/32; G06V 10/82; G06V 40/16; G16H 50/20

(57) ABSTRACT

A method performed by at least one processor includes obtaining an image of a subject; preprocessing the image of the subject; inputting the preprocessed image into a machine learning model trained in accordance with a first frequency distribution corresponding to a first ground truth obtained from one or more sensors performing a vital measurement on one or more test subjects; and obtaining, from the machine learning model, an estimate of a signal corresponding to the vital measurement of the subject.

### CROSS-REFERENCE TO RELATED APPLICATION

This application claims priority to U.S. provisional application No. 63/536,675 filed on Sep. 5, 2023, the entire contents of which are incorporated herein by reference.

### BACKGROUND

### 1. Field

This disclosure is directed to utilizing soft labels for enhanced vision-based vitals monitoring.

### 2. Related Art

Heart rate (HR) and heart rate variability (HRV) are valuable vitals, biomarkers, and physiological parameters for estimating a person's cardiac function. Most devices that measure the cardiac pulse require contact with the subject's body, such as fingertip oximeters (PPG) or electrocardiogram (ECG) patches. Additionally, measurement devices may be prohibitively expensive, resulting in measurements only being taken during visits to medical establishments.

A vision-based method for non-contact measurement of a blood volume pulse from a camera has been introduced. This vision-based method is known as remote photoplethysmography (rPPG). rPPG enables low-cost and ubiquitous health monitoring from low-cost cameras, which are readily available in mobile phones, computers, tablets, etc. The rPPG signal may be analyzed to extract multiple physiological parameters including, but not limited to, HR, HRV, RR (respiration rate), SpO2 (oxygen saturation), or BP (blood pressure). While the beneficial impacts are clear, implementing accurate rPPG systems is difficult in practice.

rPPG allows for non-contact measurement of the blood volume pulse from commodity cameras. The vast majority of research has evaluated the robustness of rPPG systems via the frequency (e.g., pulse rate in beats per minute (bpm)) over short time windows. As the systems improve, it is beneficial to support more challenging measurement configurations.

Although camera-based vitals measurements have improved over recent years, traditional rPPG methods follow step-by-step transformations from single input video to a time signal representing the pulse (rPPG). Popular methods include color transformations, blind source separation, and signal processing. These methods do not always handle noise factors (e.g., motion) in an environment. To create the most robust rPPG algorithms, researchers have begun exploring data-driven methods such as deep learning using convolutional neural networks (CNN) or transformers to predict an rPPG time signal from only the video. The neural networks are trained with supervised learning frameworks, where PPG or ECG ground truth signal is used as the target label during backpropagation.

However, for deep learning systems to be trustworthy and generalizable, the current solutions require large training datasets with a diverse set of skin tones, lighting, camera sensors, motion, and coverage of the physiological ranges. Collecting such diverse data is challenging due to the need for simultaneous capture of a physiological ground truth. Many modern deep learning frameworks for rPPG even require a time-synchronized PPG waveform.

### SUMMARY

According to an aspect of the disclosure, a method performed by at least one processor comprises obtaining an image of a subject; preprocessing the image of the subject; inputting the preprocessed image into a machine learning model trained in accordance with a first frequency distribution corresponding to a first ground truth obtained from one or more sensors performing a vital measurement on one or more test subjects; and obtaining, from the machine learning model, an estimate of a signal corresponding to the vital measurement of the subject.

According to an aspect of the disclosure, an apparatus comprises: a memory; processing circuitry coupled to the memory, the processing circuitry configured to: obtain an image of a subject, preprocess the image of the subject, input the preprocessed image into a machine learning model trained in accordance with a first frequency distribution corresponding to a first ground truth obtained from one or more sensors performing a vital measurement on one or more test subjects, and obtain, from the machine learning model, an estimate of a signal corresponding to the vital measurement of the subject.

According to an aspect of the disclosure, a non-transitory computer readable medium having instructions stored therein, which when executed by a processor cause the processor to execute a method comprising: obtaining an image of a subject; preprocessing the image of the subject; inputting the preprocessed image into a machine learning model trained in accordance with a first frequency distribution corresponding to a first ground truth obtained from one or more sensors performing a vital measurement on one or more test subjects; and obtaining, from the machine learning model, an estimate of a signal corresponding to the vital measurement of the subject.

### BRIEF DESCRIPTION OF DRAWINGS

Further features, the nature, and various advantages of the disclosed subject matter will be more apparent from the following detailed description and the accompanying drawings in which:

FIG. 1 is a diagram of an environment in which methods, apparatuses, and systems described herein may be implemented, in accordance with embodiments of the present disclosure.

FIG. 2 is a block diagram of example components of one or more devices of FIG. 1, in accordance with embodiments of the present disclosure.

FIG. 3 illustrates an example system of utilizing multi-modal soft labels for vision-based vitals monitoring, in accordance with embodiments of the present disclosure.

FIG. 4 illustrates an example block diagram for determining a loss function, in accordance with embodiments of the present disclosure.

FIG. 5 illustrates an example block diagram for training the system utilizing the multi-modal soft labels, in accordance with embodiments of the present disclosure.

FIG. 6 illustrates an example system of utilizing multi-modal soft labels for vision-based vitals monitoring, in accordance with embodiments of the present disclosure.

FIG. 7 illustrates an example system of utilizing multi-modal soft labels for vision-based vitals monitoring, in accordance with embodiments of the present disclosure.

FIG. 8 illustrates an example system of utilizing multi-modal soft labels for vision-based vitals monitoring, in accordance with embodiments of the present disclosure.

### DETAILED DESCRIPTION

The following detailed description of example embodiments refers to the accompanying drawings. The same reference numbers in different drawings may identify the same or similar elements.

The foregoing disclosure provides illustration and description, but is not intended to be exhaustive or to limit the implementations to the precise form disclosed. Modifications and variations are possible in light of the above disclosure or may be acquired from practice of the implementations. Further, one or more features or components of one embodiment may be incorporated into or combined with another embodiment (or one or more features of another embodiment). Additionally, in the flowcharts and descriptions of operations provided below, it is understood that one or more operations may be omitted, one or more operations may be added, one or more operations may be performed simultaneously (at least in part), and the order of one or more operations may be switched.

It will be apparent that systems and/or methods, described herein, may be implemented in different forms of hardware or firmware. The actual specialized control hardware used to implement these systems and/or methods is not limiting of the implementations.

Even though particular combinations of features are recited in the claims and/or disclosed in the specification, these combinations are not intended to limit the disclosure of possible implementations. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set.

No element, act, or instruction used herein should be construed as critical or essential unless explicitly described as such. Also, as used herein, the articles “a” and “an” are intended to include one or more items, and may be used interchangeably with “one or more.” Where only one item is intended, the term “one” or similar language is used. Also, as used herein, the terms “has,” “have,” “having,” “include,” “including,” or the like are intended to be open-ended terms. Further, the phrase “based on” is intended to mean “based, at least in part, on” unless explicitly stated otherwise. Furthermore, expressions such as “at least one of [A] and [B]” or “at least one of [A] or [B]” are to be understood as including only A, only B, or both A and B.

Reference throughout this specification to “one embodiment,” “an embodiment,” or similar language means that a particular feature, structure, or characteristic described in connection with the indicated embodiment is included in at least one embodiment of the present solution. Thus, the phrases “in one embodiment”, “in an embodiment,” and similar language throughout this specification may, but do not necessarily, all refer to the same embodiment.

Furthermore, the described features, advantages, and characteristics of the present disclosure may be combined in any suitable manner in one or more embodiments. One skilled in the relevant art will recognize, in light of the description herein, that the present disclosure may be practiced without one or more of the specific features or advantages of a particular embodiment. In other instances, additional features and advantages may be recognized in certain embodiments that may not be present in all embodiments of the present disclosure.

Accurately estimating blood volume pulse from video is challenging. The primary difficulty is that the pulse signal is very subtle compared to other dynamics in a captured video. If the subject moves, the rPPG signal may be very difficult to determine. Furthermore, noisy camera sensors may cause pixel changes over time, which are not representative of observed environmental changes. Another challenge is inter-subject variability, where different subjects may have higher or lower amplitudes of a peripheral rPPG signal due to physical differences in melanin concentration, microvasculature, or hair covering the skin. Creating an algorithm that may reliably determine a pulse in the presence of all these factors is an ongoing research problem.

Systems that rely only on video input may have limitations to accurately measure a wide range of vitals in uncontrolled environments. A more detailed context of input signal is needed (e.g., in time or frequency domain) to distinguish subtle physiological changes from external noises without sacrificing an operating range of the physiological parameters. For example, these limitations include a measurement of heat rate (HR) or respiration rate (RR) in only nominal/resting range of subjects when stationary, or inadequate signal quality for measuring more complicated vitals such as oxygen saturation (SpO2) or Blood Pressure (BP) which rely on more subtle changes of signal amplitude.

Current systems utilize data-driven methodologies such as supervised deep learning that require large amounts of video data with target labels. In the case of rPPG, the target is a time signal of the blood volume pulse. In the past, the target pulse or ECG signal has been collected by contact-based PPG sensors or ECG patches that are time-synchronized with the camera recording the video. However, setting up a single apparatus for video and PPG collection is prone to failure, requires expertise, and is restricted to a lab setting with a computer. This set up severely limits the environmental diversity in the training data that would be necessary for training a robust deep learning model. Moreover, relying on a single modality of video limits the context provided for estimation of physiological parameters. For example, relying on a single modality of video for inferring an rPPG signal and parameters may limit the performance of vital monitoring. Furthermore, obtaining a synchronized PPG/ECG signal for training a robust rPPG model is very unlikely. However, the “soft labels” inferred from PPG/ECG signals which are not necessarily synchronized with video are more common especially in smart wearables.

Embodiments of the present disclosure are directed to systems and methods of vision-based contactless physiological parameters monitoring. The embodiments propose a novel framework that enables the rPPG model to leverage soft vital labels from other modalities besides a camera to achieve: (i) higher measurement accuracy for wider physiological value range; (ii) extended list of vitals such as blood pressure; and (iii) adaptive model to user or environment.

According to one or more embodiments, soft labels of physiological parameter values (HR, RR, SpO2, . . . ) may be leveraged during the generation of rPPG time signals. The leverage of the soft labels from other modalities such as contact sensors (PPG/ECG) may improve the system in multiple approaches including, but not limited to: (i) rPPG predictive model enhancement using soft physiological labels (e.g., soft label-based rPPG model); (ii) physiological-aware cost function for rPPG model training (rPPG cost function); (iii) physiological-aware video augmentation for robust rPPG predictive model (e.g., rPPG video augment); (iv) rPPG Predictive Model Adaptive to User and Environment (e.g., adaptive rPPG model); (v) multi-modal rPPG predictive model with soft physiological labels (e.g., multi-modal rPPG model). One or a combination of these novel components may be used to develop a multi-modal soft label based contactless vital sign monitoring.

Embodiments of the present disclosure are directed to a method to train an enhanced deep learning (DL) model architecture for predicting a time series rPPG signal from video. Instead of actual synchronized time series signal labels (PPG/ECG), the embodiments of the present disclosure may take in soft labels of physiological parameters value provided by other sources or modalities for training. According to one or more embodiments, the soft label itself may guide training of the model to capture time series signals (e.g., rPPG) corresponding to the target physiological parameter (e.g., HR).

Embodiments of the present disclosure implement a set of novel objective cost functions that consider an intrinsic nature of the physiological signal and extracted parameters to facilitate training of the DL to achieve higher accuracy. The cost functions may formulate error functions that process the generated rPPG signal in the range of physiological parameters of target to compare with soft labels of physiological parameters. These cost functions enable the predictive model to be trained on the soft labels instead of the ground truth time series signal.

Embodiments of the present disclosure are directed to a set of video augmentation methods with corresponding soft labels to the physiologically-rich videos for training more robust DL models. The automatic video augmentation that considers the intrinsic correlation of physiological parameters and characteristics of the input video advantageously prevents overfitting and improves the generalization of the DL model in changing conditions such as lighting or video information loss.

Embodiments of the present disclosure are directed to a method in which an rPPG predictive model is adapted and fine-tuned at an inference stage to capture the new dynamics of the target user and environment. This method utilizes, for example, soft physiological labels from other sources and modalities to adapt and correct the underlying rPPG model to the new dynamics of user and environment: skin tone, lighting condition, distance, or camera settings.

Embodiments of the present disclosure are directed to a method and model architecture that utilizes physiological labels from other modalities alongside video input to generate an enhanced rPPG signal. The additional context of a physiological label from another modality helps the model to better distinguish physiological signal from external noise in the video, instead of solely relying on video. The enhanced multi-modal rPPG model and enhanced rPPG signal may facilitate higher accuracy of vital monitoring as well as enabling capturing an extended list of biomarkers in uncontrolled environments such as continuous BP or SpO2.

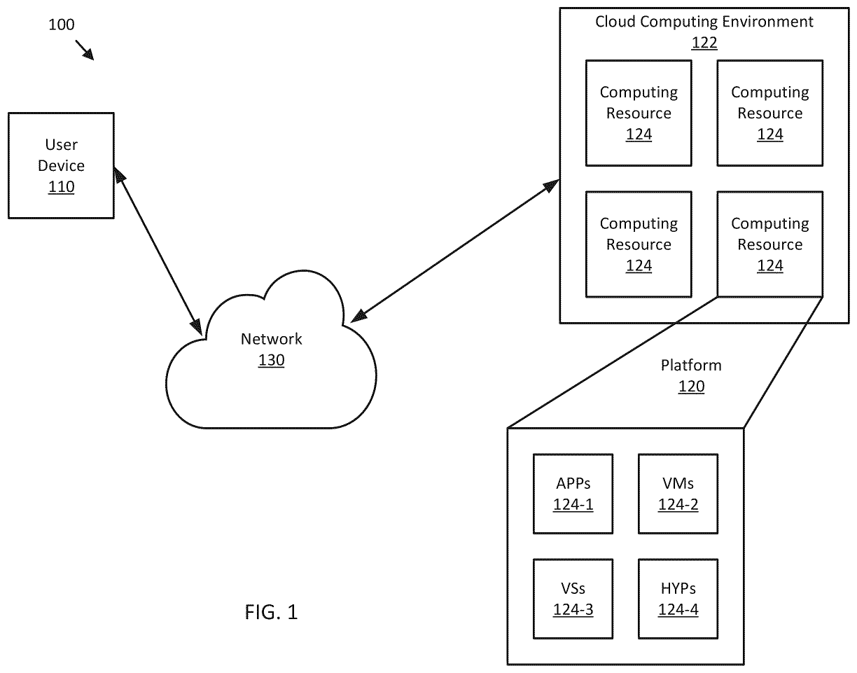

FIG. 1 is a diagram of an environment 100 in which methods, apparatuses, and systems described herein may be implemented, according to embodiments. As shown in FIG. 1, the environment 100 may include a user device 110, a platform 120, and a network 130. Devices of the environment 100 may interconnect via wired connections, wireless connections, or a combination of wired and wireless connections.

The user device 110 includes one or more devices capable of receiving, generating, storing, processing, and/or providing information associated with platform 120. For example, the user device 110 may include a computing device (e.g., a desktop computer, a laptop computer, a tablet computer, a handheld computer, a smart speaker, a server, etc.), a mobile phone (e.g., a smart phone, a radiotelephone, etc.), a wearable device (e.g., a pair of smart glasses or a smart watch), or a similar device. In some implementations, the user device 110 may receive information from and/or transmit information to the platform 120.

The platform 120 includes one or more devices as described elsewhere herein. In some implementations, the platform 120 may include a cloud server or a group of cloud servers. In some implementations, the platform 120 may be designed to be modular such that software components may be swapped in or out depending on a particular need. As such, the platform 120 may be easily and/or quickly reconfigured for different uses.

In some implementations, as shown, the platform 120 may be hosted in a cloud computing environment 122. Notably, while implementations described herein describe the platform 120 as being hosted in the cloud computing environment 122, in some implementations, the platform 120 may not be cloud-based (i.e., may be implemented outside of a cloud computing environment) or may be partially cloud-based.

The cloud computing environment 122 includes an environment that hosts the platform 120. The cloud computing environment 122 may provide computation, software, data access, storage, etc. services that do not require end-user (e.g. the user device 110) knowledge of a physical location and configuration of system(s) and/or device(s) that hosts the platform 120. As shown, the cloud computing environment 122 may include a group of computing resources 124 (referred to collectively as “computing resources 124” and individually as “computing resource 124”).

The computing resource 124 includes one or more personal computers, workstation computers, server devices, or other types of computation and/or communication devices. In some implementations, the computing resource 124 may host the platform 120. The cloud resources may include compute instances executing in the computing resource 124, storage devices provided in the computing resource 124, data transfer devices provided by the computing resource 124, etc. In some implementations, the computing resource 124 may communicate with other computing resources 124 via wired connections, wireless connections, or a combination of wired and wireless connections.

As further shown in FIG. 1, the computing resource 124 includes a group of cloud resources, such as one or more applications (APPs) 124-1, one or more virtual machines (VMs) 124-2, virtualized storage (VSs) 124-3, one or more hypervisors (HYPs) 124-4, or the like.

The application 124-1 includes one or more software applications that may be provided to or accessed by the user device 110 and/or the platform 120. The application 124-1 may eliminate a need to install and execute the software applications on the user device 110. For example, the application 124-1 may include software associated with the platform 120 and/or any other software capable of being provided via the cloud computing environment 122. In some implementations, one application 124-1 may send/receive information to/from one or more other applications 124-1, via the virtual machine 124-2.

The virtual machine 124-2 includes a software implementation of a machine (e.g. a computer) that executes programs like a physical machine. The virtual machine 124-2 may be either a system virtual machine or a process virtual machine, depending upon use and degree of correspondence to any real machine by the virtual machine 124-2. A system virtual machine may provide a complete system platform that supports execution of a complete operating system (OS). A process virtual machine may execute a single program, and may support a single process. In some implementations, the virtual machine 124-2 may execute on behalf of a user (e.g. the user device 110), and may manage infrastructure of the cloud computing environment 122, such as data management, synchronization, or long-duration data transfers.

The virtualized storage 124-3 includes one or more storage systems and/or one or more devices that use virtualization techniques within the storage systems or devices of the computing resource 124. In some implementations, within the context of a storage system, types of virtualizations may include block virtualization and file virtualization. Block virtualization may refer to abstraction (or separation) of logical storage from physical storage so that the storage system may be accessed without regard to physical storage or heterogeneous structure. The separation may permit administrators of the storage system flexibility in how the administrators manage storage for end users. File virtualization may eliminate dependencies between data accessed at a file level and a location where files are physically stored. This may enable optimization of storage use, server consolidation, and/or performance of non-disruptive file migrations.

The hypervisor 124-4 may provide hardware virtualization techniques that allow multiple operating systems (e.g. “guest operating systems”) to execute concurrently on a host computer, such as the computing resource 124. The hypervisor 124-4 may present a virtual operating platform to the guest operating systems, and may manage the execution of the guest operating systems. Multiple instances of a variety of operating systems may share virtualized hardware resources.

The network 130 includes one or more wired and/or wireless networks. For example, the network 130 may include a cellular network (e.g. a fifth generation (5G) network, a long-term evolution (LTE) network, a third generation (3G) network, a code division multiple access (CDMA) network, etc.), a public land mobile network (PLMN), a local area network (LAN), a wide area network (WAN), a metropolitan area network (MAN), a telephone network (e.g. the Public Switched Telephone Network (PSTN)), a private network, an ad hoc network, an intranet, the Internet, a fiber optic-based network, or the like, and/or a combination of these or other types of networks.

The number and arrangement of devices and networks shown in FIG. 1 are provided as an example. In practice, there may be additional devices and/or networks, fewer devices and/or networks, different devices and/or networks, or differently arranged devices and/or networks than those shown in FIG. 1. Furthermore, two or more devices shown in FIG. 1 may be implemented within a single device, or a single device shown in FIG. 1 may be implemented as multiple, distributed devices. Additionally, or alternatively, a set of devices (e.g. one or more devices) of the environment 100 may perform one or more functions described as being performed by another set of devices of the environment 100.

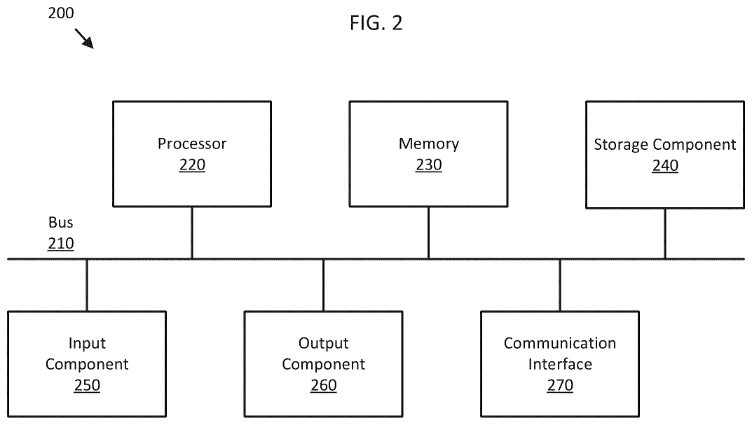

FIG. 2 is a block diagram of example components of one or more devices of FIG. 1. The device 200 may correspond to the user device 110 and/or the platform 120. The device 200 may be any other suitable device such as a TV, wall panel, etc. As shown in FIG. 2, the device 200 may include a bus 210, a processor 220, a memory 230, a storage component 240, an input component 250, an output component 260, and a communication interface 270.

The bus 210 includes a component that permits communication among the components of the device 200. The processor 220 is implemented in hardware, firmware, or a combination of hardware and software. The processor 220 is a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. In some implementations, the processor 220 includes one or more processors capable of being programmed to perform a function. The memory 230 includes a random access memory (RAM), a read only memory (ROM), and/or another type of dynamic or static storage device (e.g. a flash memory, a magnetic memory, and/or an optical memory) that stores information and/or instructions for use by the processor 220.

The storage component 240 stores information and/or software related to the operation and use of the device 200. For example, the storage component 240 may include a hard disk (e.g. a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

The input component 250 includes a component that permits the device 200 to receive information, such as via user input (e.g. a touch screen display, a keyboard, a keypad, a mouse, a button, a switch, and/or a microphone). Additionally, or alternatively, the input component 250 may include a sensor for sensing information (e.g. a global positioning system (GPS) component, an accelerometer, a gyroscope, and/or an actuator). The output component 260 includes a component that provides output information from the device 200 (e.g. a display, a speaker, and/or one or more light-emitting diodes (LEDs)).

The communication interface 270 includes a transceiver-like component (e.g., a transceiver and/or a separate receiver and transmitter) that enables the device 200 to communicate with other devices, such as via a wired connection, a wireless connection, or a combination of wired and wireless connections. The communication interface 270 may permit the device 200 to receive information from another device and/or provide information to another device. For example, the communication interface 270 may include an Ethernet interface, an optical interface, a coaxial interface, an infrared interface, a radio frequency (RF) interface, a universal serial bus (USB) interface, a Wi-Fi interface, a cellular network interface, or the like.

The device 200 may perform one or more processes described herein. The device 200 may perform these processes in response to the processor 220 executing software instructions stored by a non-transitory computer-readable medium, such as the memory 230 and/or the storage component 240. A computer-readable medium is defined herein as a non-transitory memory device. A memory device includes memory space within a single physical storage device or memory space spread across multiple physical storage devices.

Software instructions may be read into the memory 230 and/or the storage component 240 from another computer-readable medium or from another device via the communication interface 270. When executed, software instructions stored in the memory 230 and/or the storage component 240 may cause the processor 220 to perform one or more processes described herein. Additionally, or alternatively, hardwired circuitry may be used in place of or in combination with software instructions to perform one or more processes described herein. Thus, implementations described herein are not limited to any specific combination of hardware circuitry and software.

The number and arrangement of components shown in FIG. 2 are provided as an example. In practice, the device 200 may include additional components, fewer components, different components, or differently arranged components than those shown in FIG. 2. Additionally, or alternatively, a set of components (e.g. one or more components) of the device 200 may perform one or more functions described as being performed by another set of components of the device 200.

In one or more examples, the device 200 may be a controller of a smart home system that communicates with one or more sensors, cameras, smart home appliances, and/or autonomous robots. The device 200 may communicated with the cloud computing environment 122 to offload one or more tasks.

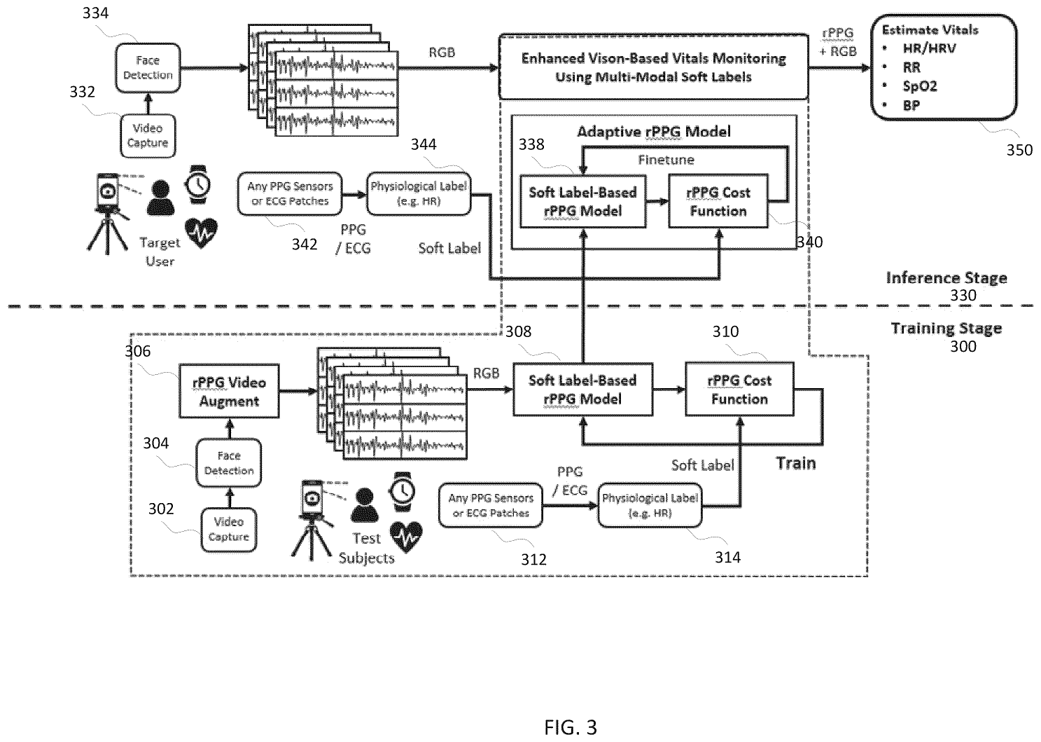

FIG. 3 illustrates an example system for utilizing multi-modal soft labels for vision-based vitals monitoring, in accordance with embodiments of the present disclosure. The system may include a training stage 300 and an inference stage 330.

In the training stage, a video capture 302 for one or more test subjects may be performed. The video capture may be performed by the user sitting in front of an electronic device containing a camera such as a smartphone, laptop, TV, etc. The video capture 302 may be a full body image or a portion of a test subject (e.g., from waist or shoulder up). After the video capture 302, a detection process may be performed to identify an area of interest. For example, a face detection 304 process may be performed in the captured image.

After face detection 304 is performed, rPPG video augment 306 may be performed. According to one or more embodiments, video augmentations are used during model training to prevent overfitting and improve the generalization of the rPPG model (e.g. in 3D CNN). For example, the video augmentation may change a video in one or more aspects to provide additional data to the model to learn.

The augmented video may be provided in the form of one or more RGB waveforms that is provided as input into a soft label-based rPPG model 308 that estimates a rPPG time series signal corresponding to an estimated vital. The output of the model 308 may be provided to a rPPG cost function 310. Furthermore, data from one or more sensors 312 may be obtained, along with a physiological label 314, which is provided as input to the rPPG cost function. In one or more examples, the data from the one or more sensors 312 may be data from PPG or ECG sensors that monitor one or more vitals of the test subjects. The rPPG cost function may calculate a loss function that indicates how closely the estimated time series signal (e.g., the output from the model 308) corresponds to the data from the one or more sensors 312.

The inference stage 330 may be used to estimate a vital of a target user. In one or more examples, the target user may be a patient that is in a telehealth medicine visit. In the inference stage, video capture 332 and face detection 334 may be performed in a similar manner as video capture 302 and face detection 304, respectively. One or more RGB signals may be input into a soft label-based rPPG model 338, which may correspond to the trained version of the model 308. In one or more examples, after the model 308 is trained, the model may be downloaded to a device of the target user, or the user may download an application that provides access to the trained model 308 stored on a server. A cost function 340 may be performed based on the output of the model 338 and a soft label 344 obtained from data of one or more sensors 342 monitoring the target user. The model 338 may be further fine-tuned based on the cost function 340. An estimate of the vitals of the target user 350 may be obtained based on the output of the model 338.

In one or more examples, video augmentations include illumination noise that adjusts a brightness of a video. By brightening or darkening the training videos, the model utilizing soft labels learns to handle different lighting in the physical environment. Since some environments may not have sufficient illumination, this video augmentation enables the model to learn to find the rPPG signal with a lower dynamic range. In one or more examples, given input video X, the augmented video X′ may be determined as follows:

$${X}^{′} = X + c , c \sim N ( 0 , σ ) , c ∈ {1}^{} \qquad Eq . ( 1 )$$

In one or more examples, video augmentations include Gaussian-pixel-wise noise. For example, camera sensors may naturally add noise to the collected video. This noise may be modeled as a Gaussian distribution independently for each pixel location and channel. Models that can still recover the rPPG signal in the presence of noise are more robust. In one or more examples, given input video X, the augmented video X′ may be determined as follows:

$${X}^{′} = X + c , c \sim N ( 0 , σ ) , c ∈ {T , H , W , C}^{} \qquad Eq . ( 2 )$$

In one or more examples, video augmentations include horizontal flipping. For example, each frame in the video may be flipped across the y-axis. This augmentation adds diversity to the spatial arrangements of face pixels during training, so the model will work with different face poses.

In one or more examples, video augmentations include random cropping. For example, a video may be randomly cropped along a width and height each from, for example, U(0.75, 1), and then linearly interpolated to the original video width and height. This augmentation simulates potential failures of the face detector, and also enables the model to isolate regions of the face that have a strong rPPG signal. In one or more examples, if only a center-cropped video is provided to the model, the model may memorize the locations of a user without attributing the actual perfusion of the video.

In one or more examples, video augmentations may include speed change where a speed of a video is modified (e.g., decrease speed or increase speed). This augmentation may be similar to the operation of random cropping, but along the time dimension of the video. In one or more examples, the video may be randomly slowed down or sped up as a factor of c, where c is sampled from U(0.75, 1.75). This augmentation may affect the underlying pulse rate of the subject. If a subject in the original video has a pulse rate of 60 bpm and the video is sped up by 1.5×, the new pulse rate will be 90 bpm. As the video's speed is changed by a factor of c, the underlying HR value may be scaled by c. This augmentation enables the model to learn a wider range of HR values than is present in the training data, thereby enabling the model to extrapolate to different physiological characteristics.

In one or more examples, video augmentations may include video encoding. For example, an rPPG signal derived from video may be affected by the information loss due to video encoding or compression. In formation loss is a common challenge in video based vital monitoring. In one or more examples, input videos may be augmented with various compression rates and encoding parameters (e.g. bit rate, profile). This augmentation enables the trained model to be more robust in reconstructing an rPPG signal despite information loss.

According to one or more embodiments, the model 308 may be a predictive rPPG model that receives RGB color signals as input and predicts a time series rPPG signal as output. The rPPG model may be trained based on a comprehensive video dataset of test subjects. In one or more examples, any suitable time series model known to one of ordinary skill in the art may be used as the architecture for the model. For example, a series of 3D CNN models may be used to capture a correlation in the time domain and spatial domain of the RGB signals in different locations of a region of interest (e.g., user's face). In one or more examples, the 3D CNN model effectively captures these correlations to reconstruct the rPPG signal. In one or more examples, ground truth physiological parameters may be provided using any wearable sensor of PPG or ECG patches. The ground truth in this component may be the “soft label” produced by the ground truth device, but does not have to be synchronized precisely with the video signal. For example, a heart rate from a PPG device may be used as the “soft label” ground truth.

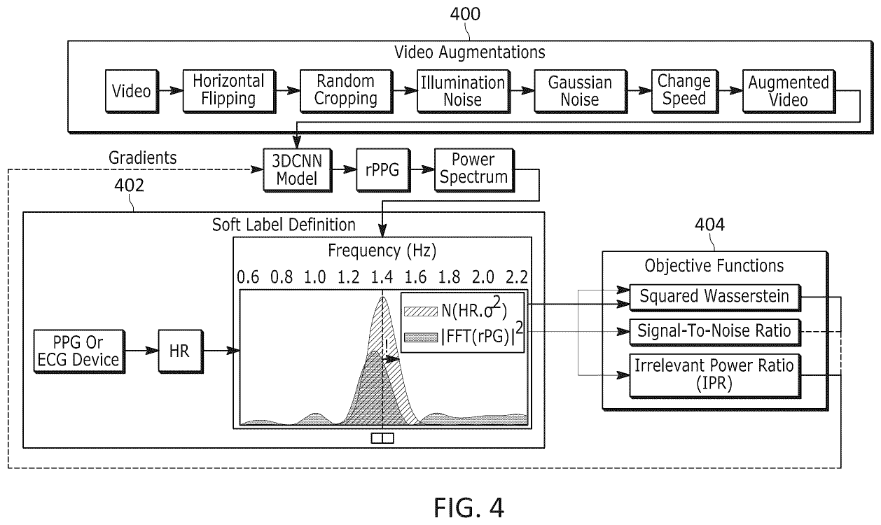

According to one or more embodiments, the ground truth HR label collected from the ground truth device may be transformed to the same domain as the rPPG model's power spectral density by defining a Gaussian distribution over the frequency bins. The Gaussian distribution may be centered at a HR frequency, u, with a standard deviation as a function of the main lobe. For example, given a video of N frames sampled at f frames per second, the standard deviation is defined as a function of the main lobe (f/N). The equation below defines the target Gaussian distribution illustrated in FIG. 4:

$$Y = N ( μ , \frac{f / N}{3} ) \qquad Eq . ( 3 )$$

As illustrated in FIG. 4, a captured video is augmented 400 using one of the video augmentation techniques discussed above. The augmented video is provided to a 3DCNN Model to predict an rPPG signal. A power spectrum of the rPPG signal may be obtained. For example, the time signal may be converted to the frequency domain with the fast Fourier transform (FFT). The power spectrum may be computed by taking the square of the absolute value of the output of the FFT. The equation below describes this process as follows:

$$\hat{Y} = {\vert FFT ( rPPG ) \vert}^{2} \qquad Eq . ( 4 )$$

In one or more examples, a soft label definition 402 may be obtained from one or more devices (e.g., PPG, ECG) measuring vitals of one or test subjects such as a heart rate. The power spectrum of the rPPG signal may be compared with the soft label definition, where one or more objective functions 404 may be used as a cost function.

In one or more examples, the predicted rPPG signal may be processed using an rPPG cost function and compared with a processed ground truth “soft label” to calculate the prediction error or gradients for training. In one or more examples, the distribution with diagonal lines in FIG. 4 shows an example of the soft frequency label, Y. Once the label and prediction are in the frequency domain together, the three objective functions (404) may be used to give feedback to the neural network for training. The first objective function may be a weakly supervised objective, which may be defined as a squared Wasserstein distance between the two distributions over the d frequency bins as follows:

$${L}_{ws} ( \hat{Y} , Y ) = \frac{1}{d} {\sum }_{i = 0}^{d} {( {CDF}_{i} ( \hat{Y} ) - {CDF}_{i} ( Y ) )}^{2} \qquad Eq . ( 5 )$$

In one or more examples, the second objective may be a signal-to-noise ratio (SNR), defined as a proportion of power centered at the peak frequency compared to the sum of power between the lower and upper cutoff frequencies (a and b, respectively) of the physiological signal as follows:

$${L}_{snr} ( \hat{Y} ) = {( {\sum }_{i = a}^{b} {\hat{Y}}_{i} )}^{- 1} ( {\sum }_{i = a}^{{\hat{Y}}^{*} - Δ d} {\hat{Y}}_{i} + {\sum }_{i = {Y}^{*} + Δ d}^{b} {\hat{Y}}_{i} ) \qquad Eq . ( 6 )$$

In one or more examples, the third objective function may be an irrelevant power ratio (IPR), defined as the proportion of power between the lower and upper cutoff frequencies of the physiological signal (a and b, respectively) compared to the total power of the signal as follows:

$${L}_{ipr} ( \hat{Y} ) = {( {\sum }_{i = - ∞}^{∞} {\hat{Y}}_{i} )}^{- 1} ( {\sum }_{i = - ∞}^{a} {\hat{Y}}_{i} + {\sum }_{i = b}^{∞} {\hat{Y}}_{i} ) \qquad Eq . ( 7 )$$

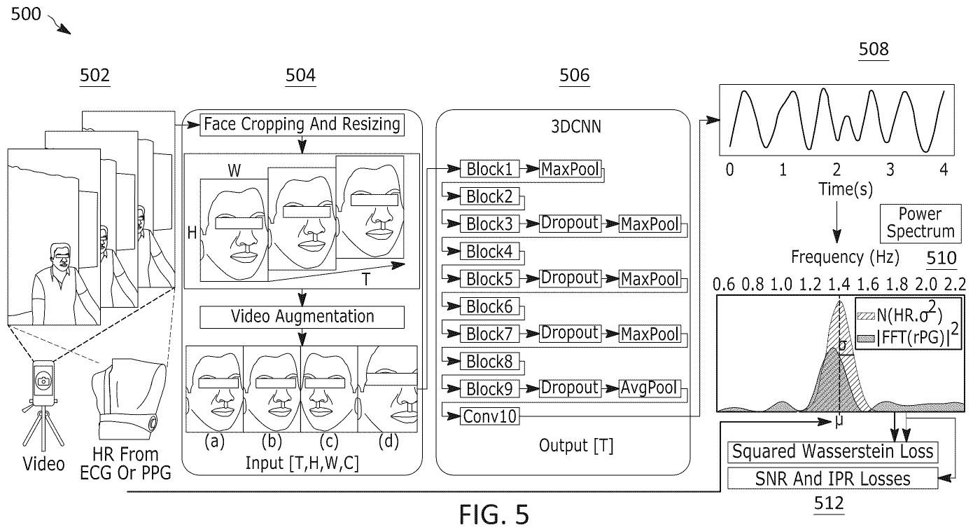

FIG. 5 illustrates an example block diagram for training the system utilizing the multi-modal soft labels, in accordance with embodiments of the present disclosure.

In a first stage 502, a video of a subject may be captured. In a second stage 504, preprocessing may be performed on the captured image. For example, to simplify the learning process for the network, images may be detected, cropped, and downscaled before each forward pass. Next, the minimum and maximum landmark locations may be identified along the x-and y-axes. The face may be cropped with padding along the edges (e.g., a 6% padding on the sides and a 22% padding on the top and bottom). After cropping the face, the image may be down sampled with bilinear interpolation to 50×70 pixels, which may be a similar aspect ratio to face dimensions. This process may be applied to all video frames, resulting in a tensor X∈RT×70×50×C, where T is the number of frames, and C is the channel dimension (e.g., RGB).

In a third stage 506, the preprocessed video is provided to the model. In one or more examples, the model is a neural network (e.g., 3D convolutional neural network). The neural network model may input the clip X (e.g., preprocessed video), and predicts a time signal with the same number of samples Y∈ [symbol] ^{T}. In stage 506, each “Block” may perform 3D convolution, batch normalization, and a ReLU activation. In stage 506, the “Conv10” layer may be a 1×1 convolutional layer to contract to a single output channel without any activation function.

The output of stage 506 may be an rPPG time signal 508 that is converted to the frequency domain. The power spectrum of the rPPG time signal may be compared with the soft label in the frequency domain in stage 510, where a loss function may be calculated in stage 512.

Each training clip may have a corresponding frequency label of the pulse rate. Since the models predict a waveform, which is then transformed to the frequency domain, the labels may be defined as Gaussian distributions centered at the ground truth pulse rates. The standard deviation of the Gaussian labels may correspond to a “softness.” As the standard deviation approaches zero, the label becomes one-soft, and as the standard deviation increases, the label becomes more over the range of supported frequencies. The standard deviation may be set as the lobe width (sampling rate divided by the number of samples) divided by integers (e.g., from 1 to 6).

In one or more examples, the model may be fine-tuned to a user's specific environment. For example, as illustrated in FIG. 3, the output of the cost function 340 is provided to the model 338 for adjusting the model 338 based on a target user's environment. Particularly, a general predictive model may not capture all variations of the user and the environment. There are numerous factors that impact the intensity of light captured from the target user, which is related to physiology. For example, a target user's skin type, ambient light intensity, light temperature, distance, etc., may affect the estimated rPPG model. The model may need a comprehensive dataset to model these variations.

According to one or more embodiments, “soft labels” are integrated with the “rPPG cost function” to perform fine-tuning or calibration of the model 338 at runtime. Based on these features, the general model (e.g., 308) that was pre-trained offline (e.g., on cloud) may be fine-tuned on the target user's device based on one or more samples of the target user's physiological parameter values from another source (e.g. wearable like smartwatch). The rPPG cost function may evaluate the error between the “soft labels” such as HR from the smartwatch and the measured vital from rPPG signal to adjust weights in the rPPG model.

According to one or more embodiments, the rPPG predictive model has specific layers and neurons tunable at the inference stage where soft labels from external sources may be used to fine-tune given the rPPG cost function. The fine-tuning of the model may be performed based on one or more of the following methods.

In one or more examples, for fine-tuning, one or more parameters corresponding to one or multiple layers of the model at upper layers are adjustable while parameters in lower layers are fixed. An additional layer may be added for “personalization” to the target user or environment of the target user. The neurons that may be tuned or adjusted may be decided automatically by an optimization algorithm based on the input, target “soft labels,” and prediction error.

In one or more examples, for fine-tuning, parameters corresponding to one or multiple layers of the model that are responsible for capturing an impact of the specific user or environment factor on an rPPG signal, may be adjustable while parameters in other layers are kept fixed. Layers and neurons that may be tunable or adjusted may be decided offline based on the analysis of the rPPG model. For example, an adjustment to light temperature may be performed at middle layers of the model where the correlation between RGB frequency features are captured to reconstruct the rPPG signal. In one or more examples, when calibrating for user skin factors, lower-to-mid layers of the model where the correlation of RGB color intensity signal is being captured to distinguish physiological signal vs noise, may be adjusted.

The model may be optimized occasionally with available sporadic “soft label” samples from a ground truth device. The triggering event for fine-tuning a model may be dependent on a prediction error, user/environment factor changes, or based on a predetermined timing (e.g., model fine-tuned every hour). In one or more examples, reinforcement learning methods may be utilized to help speed up the calibration of the personalized model.

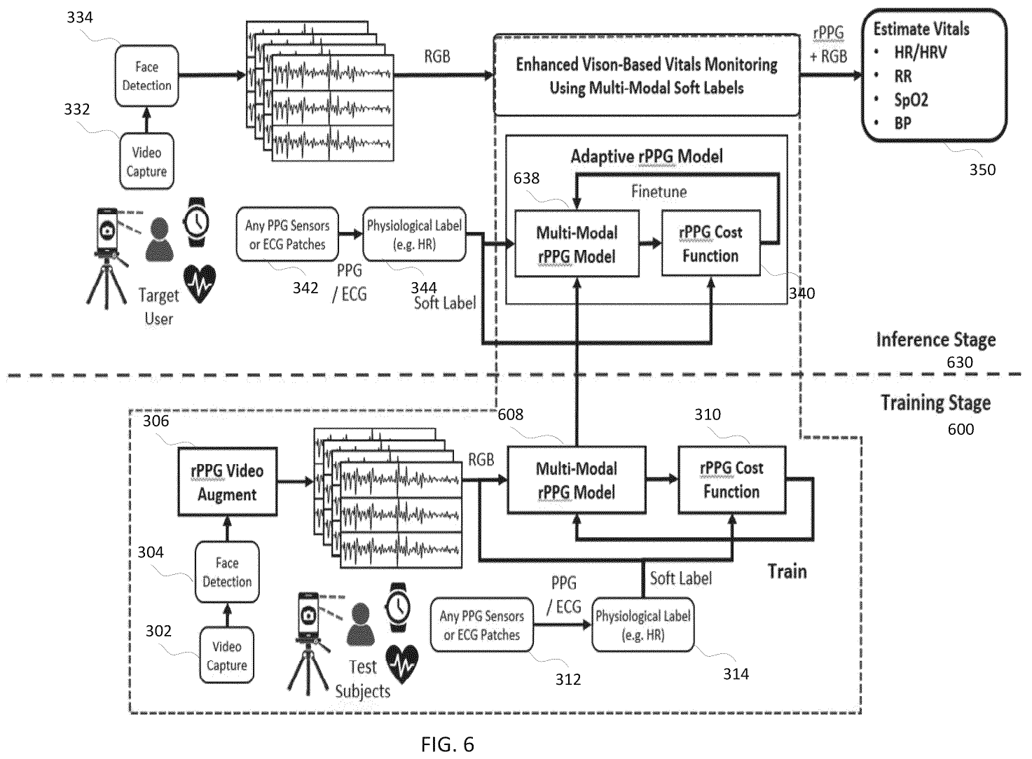

FIG. 6 illustrates an example system of utilizing multi-modal soft labels for vision-based vitals monitoring, in accordance with embodiments of the present disclosure. Descriptions of components common with the system illustrated in FIG. 3 are not repeated.

The system of FIG. 6 includes a training stage 600 and an inference stage 630. The training stage 600 pre-trains a multi-modal rPPG model 608, and the inference stage 630 utilizes and fine-tunes a multi-modal rPPG model 638. The model 638 may correspond to the pre-trained model 608 before fine-tuning is performed.

According to one or more embodiments, “soft labels” from an external source may be utilized as additional context in the rPPG model (e.g., model 608 and model 638). The additional context aids as another modality to distinguish between a physiological signal and a noise signal in the video. The “soft label” may be converted to a synthetic time series physiological signal. This converted signal may be a reference signal for the model to use when converting RGB channels to the rPPG signal. In one or more examples, when the external source provides a raw signal PPG or ECG, these signals may also be used as the additional modality.

As illustrated in FIG. 6, in one or more examples, the same architecture may be used for the multi-modal rPPG model (e.g., model 608 and model 638) as the single-modal model (e.g., model 308 and model 338). In one or more examples, a channel corresponding to a physiological signal may be combined by other RGB channels and fed to a 3D CNN. If more sources are available, more channels may be appended to each other.

In one or more examples, a target of the model training may be similar to the previous components. The “soft label” may be used again to create a distribution of the physiological values to evaluate error with the rPPG-based physiological parameter.

At the inference stage 630, additional sources of data may be used to generate an enhanced rPPG signal for vital monitoring, resulting in a wider range of values or extended list of physiological parameters.

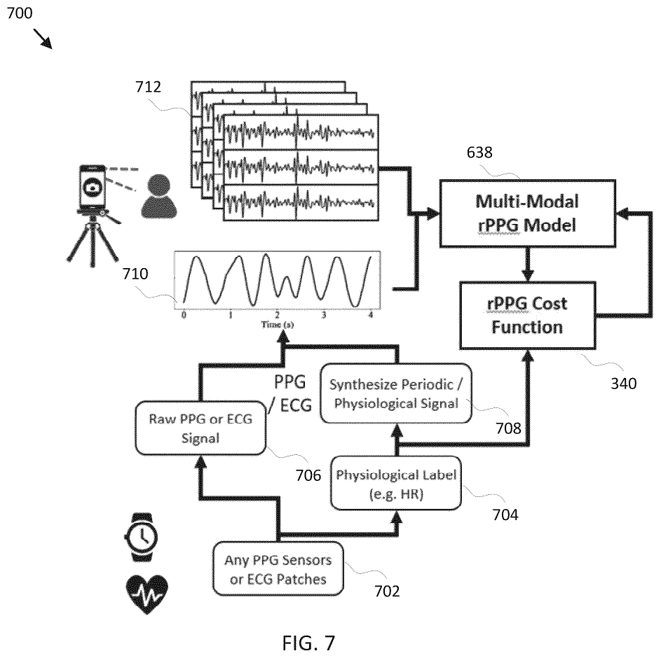

FIG. 7 illustrates an example system of utilizing multi-modal soft labels for vision-based vitals monitoring, in accordance with embodiments of the present disclosure. In the system 700, data from one or more PPG sensors or ECG patches 702 may be collected. This data may be from a target user, or data from other users other than the target user. A physiological label 704 and raw PPG or ECG signal 706 may be determined from the data from the PPG sensors or ECG patches. Furthermore, a periodic/physiological signal 708 may be synthesized from the physiological label 704. A time series signal 710 and RGB data 712 corresponding to a video capture of a target user may be provided to the model 638. The physiological label 704 and output of the model 638 may be provided to the cost function 340. Furthermore, the output of the cost function 340 may be used to fine-tune the model 638.

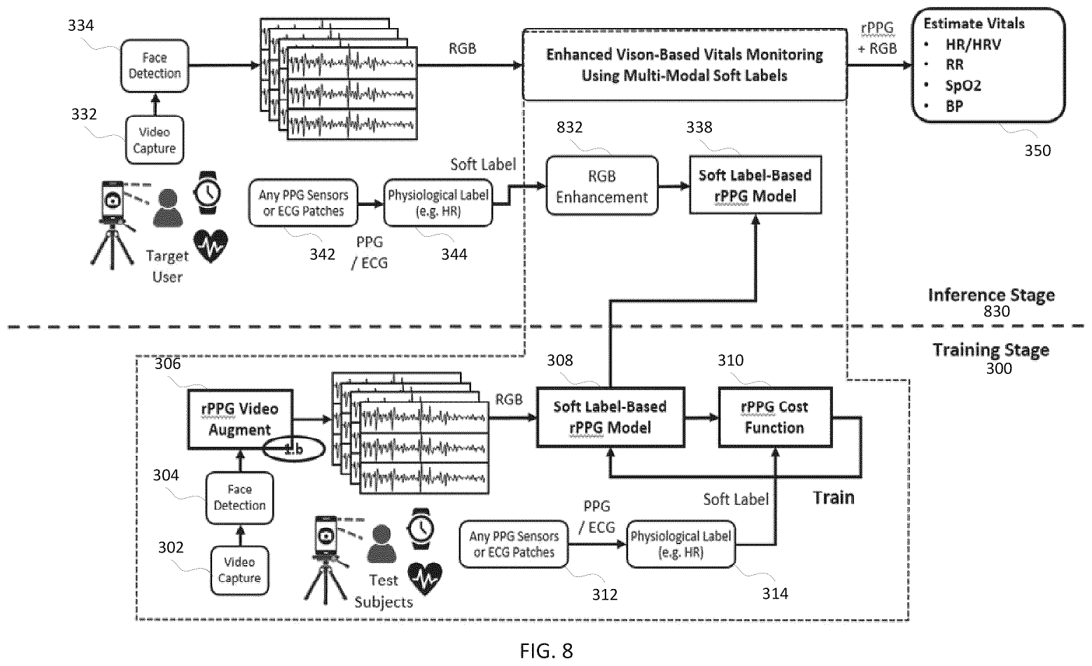

FIG. 8 illustrates an example system of utilizing multi-modal soft labels for vision-based vitals monitoring, in accordance with embodiments of the present disclosure. The system in FIG. 8 includes the training stage 300 of FIG. 3 and an inference stage 830. In the inference stage 830, additional context from an external source may be utilized to pre-process RGB signals before utilizing in the rPPG model to predict the final rPPG signal. For example, another source measuring samples of HR or RR can provide additional context regarding the narrower range of user's vital at the moment. This additional context may help filter the RGB signals to a more specific range corresponding to the physiological signal, excluding noisy signals. Without the additional context, there is no prior information regarding the range of physiological parameters values; hence, the method may sacrifice accuracy of measurement to look for a physiological signal in all possible ranges.

In one or more examples, in RGB enhancement 832, various methods of filtering, bandpass filter, Kalman filter, etc. may be utilized in the preprocessing stage before feeding RGB signal to the rPPG model 338. In one or more examples, another ML model may be also developed for the purpose of rPPG enhancement using the additional source of physiological value. Combining the above steps of rPPG enhancement within one single model of rPPG model results in the “multi-modal” rPPG model as discussed above.

The embodiments have been described above and illustrated in terms of blocks, as shown in the drawings, which carry out the described function or functions. These blocks may be physically implemented by analog and/or digital circuits including one or more of a logic gate, an integrated circuit, a microprocessor, a microcontroller, a memory circuit, a passive electronic component, an active electronic component, an optical component, and the like, and may also be implemented by or driven by software and/or firmware (configured to perform the functions or operations described herein). The circuits may, for example, be embodied in one or more semiconductor chips, or on substrate supports such as printed circuit boards and the like. Circuits included in a block may be implemented by dedicated hardware, or by a processor (e.g., one or more programmed microprocessors and associated circuitry), or by a combination of dedicated hardware to perform some functions of the block and a processor to perform other functions of the block. Each block of the embodiments may be physically separated into two or more interacting and discrete blocks. Likewise, the blocks of the embodiments may be physically combined into more complex blocks.

While this disclosure has described several non-limiting embodiments, there are alterations, permutations, and various substitute equivalents, which fall within the scope of the disclosure. It will thus be appreciated that those skilled in the art will be able to devise numerous systems and methods which, although not explicitly shown or described herein, embody the principles of the disclosure and are thus within the spirit and scope thereof.

According to one or more embodiments, a method includes: accessing a training dataset comprising videos of test subjects' faces and corresponding sensor data (e.g., from PPG or ECG sensor), where the videos and the sensor data are not time-synchronized; determining Gaussian distributions based on the sensor data to be used as soft labels for training a machine learning model; providing the videos of test subjects' faces as input to the machine learning model configured to predict rPPG signals from the videos; and training a machine learning model using a loss function that includes a difference between the Gaussian distribution based on the sensor data and FFT of the predicted rPPG.

The method further includes: during the inference phase, receiving sensor data (e.g., heart rate measured using a smartwatch) collected while a user is capturing a video for rPPG prediction; fine tuning the machine learning model to be adapted for the user by updating parameters in one or more layers of the machine learning model, where the Gaussian distribution determined from the sensor data is used as soft labels for the predicted rPPG.

The above disclosure also encompasses the embodiments listed below:
(1) A method performed by at least one processor, the method including: obtaining an image of a subject; preprocessing the image of the subject; inputting the preprocessed image into a machine learning model trained in accordance with a first frequency distribution corresponding to a first ground truth obtained from one or more sensors performing a vital measurement on one or more test subjects; and obtaining, from the machine learning model, an estimate of a signal corresponding to the vital measurement of the subject.
(2) The method according to feature (1), further including: obtaining, from one or more sensors performing the vital measurement on the subject, a second frequency distribution corresponding to a second ground truth of the vital measurement; converting the estimate of the signal corresponding to the vital measurement of the subject to a frequency domain signal; determining an error between the frequency domain signal and the second frequency distribution; and updating the machine learning model based on the determined error.
(3) The method according to feature (1) or (2), in which the first frequency distribution is a Gaussian distribution centered at the first ground truth and having a first standard deviation that is a function of a N frames sampled a f frames per second, in which N and f are positive integers.
(4) The method according to feature (2) or (3), in which the first frequency distribution is a Gaussian distribution centered at the first ground truth and having a second standard deviation that is a function of a N frames sampled a f frames per second, in which N and f are positive integers.
(5) The method according to feature (3) or (4), in which the determining the error includes: determining a mean squared error (MSE) loss between the frequency domain signal and the second frequency distribution.
(6) The method according to feature (5), in which the determining the error further includes: determining a signal-to-noise ratio (SNR) based on a proportion of a power centered at a peak frequency of the frequency domain signal compared to a sum of power between a lower cutoff frequency and an upper cutoff frequency of the frequency domain signal.
(7) The method according to feature (6), in which the determining the error further includes: determining an irrelevant power ratio (IPR) based on a proportion of a power between the lower cutoff frequency and the upper cutoff frequency of the frequency domain signal compared to a total power of the frequency domain signal.
(8) The method according to any one of features (1)-(7), in which the preprocessing the image of the subject comprises: detecting a region of interest of the image subject; and resizing the region of interest of the image subject.
(9) The method of feature (8), in which the region of interest is at least a portion of a face of the subject.
(10) The method according to any one of features (1)-(9), in which the vital measurement is one of a pulse rate, blood pressure, oxygen saturation level.
(11) The method according to any one of features (1)-(10) in which the machine learning model is a three dimensional (3D) Convolutional Neural Network (CNN).
(12) An apparatus including: a memory; processing circuitry coupled to the memory, the processing circuitry configured to obtain an image of a subject, preprocess the image of the subject, input the preprocessed image into a machine learning model trained in accordance with a first frequency distribution corresponding to a first ground truth obtained from one or more sensors performing a vital measurement on one or more test subjects, and obtain, from the machine learning model, an estimate of a signal corresponding to the vital measurement of the subject.
(13) The apparatus according to feature (12), in which the processing circuitry is further configured to: obtain, from one or more sensors performing the vital measurement on the subject, a second frequency distribution corresponding to a second ground truth of the vital measurement, convert the estimate of the signal corresponding to the vital measurement of the subject to a frequency domain signal, determine an error between the frequency domain signal and the second frequency distribution, and update the machine learning model based on the determined error.
(14) The apparatus according to feature (12) or (13), in which the first frequency distribution is a Gaussian distribution centered at the first ground truth and having a first standard deviation that is a function of a N frames sampled a f frames per second, in which N and f are positive integers.
(15) The apparatus according to feature (13) or (14), in which the first frequency distribution is a Gaussian distribution centered at the first ground truth and having a second standard deviation that is a function of a N frames sampled a f frames per second, in which N and f are positive integers.
(16) The apparatus according to feature (14) or (15), in which the processing circuitry, to determine the error, is further configured to: determine a mean squared error (MSE) loss between the frequency domain signal and the second frequency distribution.
(17) The apparatus according to feature (16), in which the processing circuitry, to determine the error, is further configured to: determine a signal-to-noise ratio (SNR) based on a proportion of a power centered at a peak frequency of the frequency domain signal compared to a sum of power between a lower cutoff frequency and an upper cutoff frequency of the frequency domain signal.
(18) The apparatus according to feature (17), in which the processing circuitry, to determine the error, is further configured to: determine an irrelevant power ratio (IPR) based on a proportion of a power between the lower cutoff frequency and the upper cutoff frequency of the frequency domain signal compared to a total power of the frequency domain signal.
(19) The apparatus according to any one of features (12)-(18), in which the processing circuitry, to preprocess the image of the subject, is further configured to: detect a region of interest of the image subject, and resize the region of interest of the image subject.
(20) A non-transitory computer readable medium having instructions stored therein, which when executed by a processor cause the processor to execute a method including: obtaining an image of a subject; preprocessing the image of the subject; inputting the preprocessed image into a machine learning model trained in accordance with a first frequency distribution corresponding to a first ground truth obtained from one or more sensors performing a vital measurement on one or more test subjects; and obtaining, from the machine learning model, an estimate of a signal corresponding to the vital measurement of the subject.

What is claimed is:

1. A method performed by at least one processor, the method comprising:
obtaining an image of a subject;
preprocessing the image of the subject;
inputting the preprocessed image into a machine learning model trained in accordance with a first frequency distribution corresponding to a first ground truth obtained from one or more sensors performing a vital measurement on one or more test subjects; and
obtaining, from the machine learning model, an estimate of a signal corresponding to the vital measurement of the subject.

2. The method according to claim 1, further comprising:
obtaining, from one or more sensors performing the vital measurement on the subject, a second frequency distribution corresponding to a second ground truth of the vital measurement;
converting the estimate of the signal corresponding to the vital measurement of the subject to a frequency domain signal;
determining an error between the frequency domain signal and the second frequency distribution; and
updating the machine learning model based on the determined error.

3. The method according to claim 1, wherein the first frequency distribution is a Gaussian distribution centered at the first ground truth and having a first standard deviation that is a function of a N frames sampled a f frames per second, wherein N and f are positive integers.

4. The method according to claim 2, wherein the first frequency distribution is a Gaussian distribution centered at the first ground truth and having a second standard deviation that is a function of a N frames sampled a f frames per second, wherein N and f are positive integers.

5. The method according to claim 3, wherein the determining the error comprises:
determining a mean squared error (MSE) loss between the frequency domain signal and the second frequency distribution.

6. The method according to claim 5, wherein the determining the error further comprises:
determining a signal-to-noise ratio (SNR) based on a proportion of a power centered at a peak frequency of the frequency domain signal compared to a sum of power between a lower cutoff frequency and an upper cutoff frequency of the frequency domain signal.

7. The method according to claim 6, wherein the determining the error further comprises:
determining an irrelevant power ratio (IPR) based on a proportion of a power between the lower cutoff frequency and the upper cutoff frequency of the frequency domain signal compared to a total power of the frequency domain signal.

8. The method of claim 1, wherein the preprocessing the image of the subject comprises:
detecting a region of interest of the image subject; and
resizing the region of interest of the image subject.

9. The method of claim 8, wherein the region of interest is at least a portion of a face of the subject.

10. The method of claim 1, wherein the vital measurement is one of a pulse rate, blood pressure, oxygen saturation level.

11. The method of claim 1 wherein the machine learning model is a three dimensional (3D) Convolutional Neural Network (CNN).

12. An apparatus comprising:
a memory;
processing circuitry coupled to the memory, the processing circuitry configured to:
obtain an image of a subject,
preprocess the image of the subject,
input the preprocessed image into a machine learning model trained in accordance with a first frequency distribution corresponding to a first ground truth obtained from one or more sensors performing a vital measurement on one or more test subjects, and
obtain, from the machine learning model, an estimate of a signal corresponding to the vital measurement of the subject.

13. The apparatus according to claim 12, wherein the processing circuitry is further configured to:
obtain, from one or more sensors performing the vital measurement on the subject, a second frequency distribution corresponding to a second ground truth of the vital measurement,
convert the estimate of the signal corresponding to the vital measurement of the subject to a frequency domain signal,
determine an error between the frequency domain signal and the second frequency distribution, and
update the machine learning model based on the determined error.

14. The apparatus according to claim 12, wherein the first frequency distribution is a Gaussian distribution centered at the first ground truth and having a first standard deviation that is a function of a N frames sampled a f frames per second, wherein N and f are positive integers.

15. The apparatus according to claim 13, wherein the first frequency distribution is a Gaussian distribution centered at the first ground truth and having a second standard deviation that is a function of a N frames sampled a f frames per second, wherein N and f are positive integers.

16. The apparatus according to claim 14, wherein the processing circuitry, to determine the error, is further configured to:
determine a mean squared error (MSE) loss between the frequency domain signal and the second frequency distribution.

17. The apparatus according to claim 16, wherein the processing circuitry, to determine the error, is further configured to:
determine a signal-to-noise ratio (SNR) based on a proportion of a power centered at a peak frequency of the frequency domain signal compared to a sum of power between a lower cutoff frequency and an upper cutoff frequency of the frequency domain signal.

18. The apparatus according to claim 17, wherein the processing circuitry, to determine the error, is further configured to:
determine an irrelevant power ratio (IPR) based on a proportion of a power between the lower cutoff frequency and the upper cutoff frequency of the frequency domain signal compared to a total power of the frequency domain signal.

19. The apparatus according to claim 12, wherein the processing circuitry, to preprocess the image of the subject, is further configured to:
detect a region of interest of the image subject, and
resize the region of interest of the image subject.

20. A non-transitory computer readable medium having instructions stored therein, which when executed by a processor cause the processor to execute a method comprising:
obtaining an image of a subject;
preprocessing the image of the subject;
inputting the preprocessed image into a machine learning model trained in accordance with a first frequency distribution corresponding to a first ground truth obtained from one or more sensors performing a vital measurement on one or more test subjects; and
obtaining, from the machine learning model, an estimate of a signal corresponding to the vital measurement of the subject.
